(19) United States
(12) Patent Application Publication
(10) Pub. No.: US 2025/0195389 A1
(43) Pub. Date: Jun. 19, 2025

(54) MINERAL SUNSCREEN COMPOSITION COMPRISING HYDROPHOBIC POLYMER

(71) Applicant: L'OREAL, Paris (FR)

(72) Inventors: Ritesh Kumar SINHA, Linden, NJ (US); Hy Si BUI, Piscataway, NJ (US); Mohamed KANJI, Edison, NJ (US); Xuefei LIU, Edison, NJ (US)

(73) Assignee: L'OREAL, Paris (FR)

(21) Appl. No.: 18/544,283

(22) Filed: Dec. 18, 2023

(51) Int. Cl. A61K 8/81; A61K 8/06; A61K 8/19; A61Q 17/04

(52) U.S. Cl. CPC A61K 8/8147; A61K 8/062; A61K 8/19; A61Q 17/04; A61K 2800/596

(57) ABSTRACT

Mineral sunscreen compositions are described, having (a) a hydrophobic polymer formed as a reaction product of a natural or food-derived oil and a methacrylate or acrylate polymer; (b) one or more solvents capable of solubilizing the hydrophobic polymer; (c) one or more mineral UV filtering agents; (d) one or more surfactants; and (e) water; wherein the composition is an oil-in-water emulsion. Methods from making and using the mineral sunscreen compositions are also described.

### FIELD OF THE DISCLOSURE

The present disclosure relates to mineral sunscreen compositions comprising a unique hydrophobic polymer, and to methods for using the mineral sunscreen compositions to protect skin and hair from ultraviolet light.

### BACKGROUND

The negative effects of exposure to ultraviolet (“UV”) light are well known. Prolonged exposure to sunlight causes damage such as sunburn to the skin and dries out hair making it brittle. When skin is exposed to UV light having a wavelength of from about 290 nm to about 400 nm, long term damage can lead to serious conditions such as skin cancer.

UV light also contributes to aging by causing free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. Damage to the cell membrane results in loss of cell permeability, increased intercellular ionic concentration, and decreased ability to excrete or detoxify waste products. The end result is a loss of skin elasticity and the appearance of wrinkles. This process is commonly referred to as photo-aging.

Sunscreens can be used to protect against UV damage and delay the signs of aging. The degree of UV protection afforded by a sunscreen composition is directly related to the amount and type of UV filters contained therein. The higher the amount of UV filters, the greater the degree of UV protection. Mineral UV filtering agents often produce a white color when applied to the skin, especially when used in high amounts in sunscreen formulations, which many consumers dislike. Also, consumers seek sunscreen composition that are water resistant to avoid having to reapply the sunscreen composition often, especially when enjoying the water. The inventors of the instant disclosure sunscreen compositions that improve the transparency of mineral sunscreen composition, are water-resistant, and provide exceptional protection from harmful UV rays.

### SUMMARY OF THE INVENTION

The instant disclosure relates to sunscreen compositions that are highly effective, water-resistant, and aesthetically pleasing. The sunscreen compositions include mineral UV filtering agents, which are known to be non-irritating, natural, and gentle to the skin. One drawback with mineral-based sunscreen compositions, however, is the tendency to exhibit a white appearance on the skin. Consumers prefer sunscreen compositions to appear natural (unnoticeable). Developing mineral-based sunscreen products having a high Sun Protection Factor (SPF) that exhibit minimal whitening, however, is challenging. The inventors discovered that use of a hydrophobic polymer formed as a reaction product of a natural or food-derived oil and a methacrylate or acrylate polymer not only improves transparency of mineral-based sunscreen composition but also boosts water-resistance and durability. The sunscreen compositions are long-lasting, withstand washing, and minimize whitening. Typically, the mineral sunscreen compositions are oil-in-water emulsions and include:
(a) a hydrophobic polymer formed as a reaction product of a natural or food-derived oil and a methacrylate or acrylate polymer;
(b) one or more solvents capable of solubilizing the reaction product of (a);
(c) one or more mineral UV filtering agents;
(d) one or more surfactants; and
(e) water.

The hydrophobic polymer can be the reaction product of a natural or food-derived oil and an acrylate or methacrylate polymer. Preferably, the the hydrophobic polymer is a reaction product of a natural or food-derived oil and a methacrylate polymer. The natural or food-derived oil may be a drying oil or a semi-drying oil. Nonlimiting examples include linseed oil, sunflower oil, tung oil, fish oil, cottonseed oil, soybean oil, or combinations thereof. The methacrylate polymer can be formed from methacrylate monomers, for example, monomers selected from isobutyl methacrylate, methyl methacrylate, ethyl methacrylate, n-butyl methacrylate, and combinations thereof. In a preferred embodiment, the hydrophobic polymer is formed from a natural or food-derived oil and an isobutyl methacrylate polymer and may preferably be an isobutyl methacrylate polymer.

In various embodiments, the hydrophobic polymer is the reaction product of about 50 to about 85 parts by weight of the natural or food-derived oil and about 15 to about 50 parts by weight of the methacrylate or acrylate polymer. More specifically, the hydrophobic polymer may be the reaction product of about 72 to about 77 parts by weight of the natural or food-derived oil and about 23 to about 28 parts by weight of a methacrylate polymer. For example, the hydrophobic polymer may be the reaction product of linseed oil and poly (isobutyl methacrylate) in a suitable solvent, such as, for example, 2,2,4-trimethyl-1, 3-pentanediol monoisobutyrate. Preferably, the reaction product is formed from about 72 to about 77% of linseed oil and about 23 to about 28% of isobutyl methacrylate polymer in a suitable solvent, such as 2,2,4-trimethyl-1, 3-pentanediol monoisobutyrate.

One or more solvents capable of solubilizing the hydrophobic polymer of (a) is used to solubilize the hydrophobic polymer. A single solvent may be used or a combination of solvents, wherein the combination of solvents is capable of solubilizing the hydrophobic polymer of (a). In various embodiments, the one or more solvents capable of solubilizing the reaction product of (a) have a hydrogen bonding component (D), a polar component (P), and a dispersion component (H), and a distance (Ra) of the Hansen solubility parameter less than or equal to 13.4 MPa^{0.5}, wherein the Hansen solubility parameter is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.8 MPa^{0.5},
P₁ is 4.8 MPa^{0.5}, and
H₁ is 13.0 MPa^{0.5}.

Nonlimiting examples of solvents capable of solubilizing the hydrophobic polymer of (a) include polycitronellol acetate, caprylic/capric triglyceride, isododecane, isohexadecane, tetradecane, isopropyl myristate, isopropyl alcohol, octyldodecanol, ethanol, phenoxyethanol, castor oil, and mixtures thereof. Polycitrnoellol acetate and caprylic/capric triglyceride are particularly useful.

Nonlimiting examples of mineral UV filtering agents include treated or untreated metal oxides such as, for example, pigments or nanopigments of titanium oxide (amorphous or crystallized in rutile and/or anatase form), of iron oxide, of zinc oxide, of zirconium oxide or of cerium oxide. Particularly preferred mineral UV filtering agents include titanium dioxide and/or zinc oxide.

Surfactants include anionic surfactants, cationic surfactants, amphoteric (zwitterionic) surfactants, and nonionic surfactants. In various embodiments, the compositions of the instant disclosure include one or more anionic surfactants, and optionally, one or more nonionic surfactants. Furthermore, one or more of the surfactants maybe a biosurfactant. Nonlimiting examples of biosurfactants include glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes. Preferably, at least one of the biosurfactants is a glycolipid. Nonlimiting examples of glycolipids include sophorolipids, rhamnolipids, trehalose lipids, mannosylerythritol lipids, and combinations thereof. Rhamnolipids are particularly preferred.

Nonlimiting examples of anionic surfactants include glutamates, acyl taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates, or combinations thereof. In various embodiments, the compositions of the instant disclosure include one or more acyl taurate surfactants.

In addition to the mineral sunscreen compositions described herein, the present disclosure relates to methods for protecting skin and hair from ultraviolet (UV) radiation and to methods of absorbing ultraviolet light using the mineral sunscreen compositions. Such methods comprise application of a sunscreen composition to skin or hair. Additionally, methods for improving transparency of mineral sunscreen compositions and imparting water-resistance to mineral sunscreen compositions is described.

### DETAILED DESCRIPTION

The mineral sunscreen compositions include a hydrophobic polymer formed as a reaction product of a natural or food-derived oil and a methacrylate or acrylate polymer. The unique hydrophobic polymer unexpectedly reduces the whiteness of mineral sunscreen compositions, improves water-resistance, and contributes to formation of robust and stable emulsions. The mineral sunscreen compositions include:
(a) a hydrophobic polymer formed as a reaction product of a natural or food-derived oil and a methacrylate or acrylate polymer;
(b) one or more solvents capable of solubilizing the reaction product of (a);
(c) one or more mineral UV filtering agents;
(d) one or more surfactants;
(e) water.

The mineral sunscreen compositions are typically oil-in-water emulsions or dispersions.

### (a) Hydrophobic Polymer

The hydrophobic polymer is a reaction product of a natural or food-derived oil (oil component) and an acrylate component. In particular, the natural or food-derived oil may be a drying oil, preferably linseed oil. The reaction product may include an isobutyl methacrylate backbone with a plurality of linseed oil side chains. Preferably, the reaction product is a product sold under the MYCELX® brand from MYCELX Technologies Corporation of Gainesville, Georgia. See U.S. Pat. No. 5,698,139 for a description of MYCELX materials, which is incorporated herein by reference in its entirety.

The hydrophobic polymer is comprised of an oil component and a polymer component, typically reacted in a solvent. In a preferred embodiment, the hydrophobic polymer is a reaction product of linseed oil, poly (isobutyl methacrylate), and 2, 2, 4-trimethyl-1,3-pentanediol-monoisobutyrate as a solvent.

The oil component is derived from glycerin and carboxylic acids, such as linseed fatty acid to form monoglycerides, diglycerides, and triglycerides. The oil component is preferably derived from plant/vegetable or natural origin. Vegetable oils are obtained by cold pressing the seeds of a plant to obtain the oil contained therein. Of the vegetable oils, drying oils such as linseed and tung oil, semi-drying oils such as soybean and cotton seed oil, and non-drying oils such as coconut oil may be used as the oil component.

The polymer component may be derived from a and B-unsaturated carbonyl compounds. The polymer component is the resultant product of a monomer which is an ester of an acrylic acid, crotonic acid, isocrotonic acid, methacrylic acid, sorbic acid, cinnamic acid, maleic acid, fumaric acid, and methyl methacrylic acid. Nonlimiting examples of useful polymers which cover any number of reaction possibilities between the esters of such compounds include acrylate polymers, methyl methacrylate polymers, methyl/n-butyl methacrylate polymers, methacrylate copolymers, ethyl methacrylate, n-butyl methacrylate, isobutyl methacrylate, n-butyl/isobutyl methacrylate copolymers, or combinations thereof.

Preferably the polymer is poly (isobutyl methacrylate). In particular, the polymer percentage may be from about 23% to about 28%, or about 25.28%, of the hydrophobic polyer, e.g., poly (linseed oil/isobutyl methacrylate). The hydrophobic polymer is a reaction product typically formed in a liquid solvent able to dissolve or dilute the polymer component (poly (oil/polymer)) and the hydrophobic polymer. The solvent, or diluent should generally comprise any liquid or mixture of liquids that is able to dissolve or dilute hydrophobic polymer product. The solvent/diluent can control the evaporation, desired flow, and coalescing of the hydrophobic polymer. The solvent may be, for example, an aliphatic hydrocarbon, aromatic hydrocarbon, alcohols, ketones, ethers, aldehydes, phenols, carboxylic acids, carboxylates, synthetic chemicals and naturally occurring substances. Preferably the solvent is 2, 2, 4-trimethyl-1,3-pentanediol-monoisobutyrate. Hydrophobic polymers according to the instant disclosure and methods for making them are described, for example, in U.S. Pat. Nos. 5,437,793, 5,698,139, 5,837,146, 5,961,823, 6,180,010, 6,475,393, and 6,805,727, which are incorporated herein by reference in their entireties. The preferred hydrophobic polymer may be designated as poly (linseed oil/isobutyl methacrylate).

The amount of hydrophobic polymer in the mineral sunscreen composition will vary but may be from about 0.1 to about 15 wt. %, based on the total weight of the mineral sunscreen composition. In further embodiments, the total amount of the hydrophobic polymer in the mineral sunscreen composition is from about 0.1 to about 12 wt. %, about 0.1 to about 10 wt. %, about 0.1 to about 8 wt. %, about 0.1 to about 5 wt. %, about 0.1 to about 3 wt. %, about 0.5 to about 15 wt. %, about 0.5 to about 12 wt. %, about 0.5 to about 10 wt. %, about 0.5 to about 8 wt. %, about 0.5 to about 5 wt. %, about 0.5 to about 3 wt. %, about 1 to about 15 wt. %, about 1 to about 12 wt. %, about 1 to about 10 wt. %, about 1 to about 8 wt. %, about 1 to about 5 wt. %, about 1 to about 3 wt. %, about 2 to about 15 wt. %, about 2 to about 12 wt. %, about 2 to about 10 wt. %, about 2 to about 8 wt. %, about 2 to about 5 wt. %, based on the total weight of the mineral sunscreen composition.

### (b) Solvent Capable of Solubilizing (a)

The hydrophobic polymer is typically solubilized using one or more solvents capable of solubilizing the hydrophobic polymer. The solvent may include one or more solvents used to generate the hydrophobic polymer, e.g., 2, 2, 4-trimethyl-1,3-pentanediol-monoisobutyrate. The solvent may be a single solvent or a plurality of solvents. For example, in various embodiments, solvents capable of solubilizing the hydrophobic polymer of (a) have a dispersion component (D), a polar component (P), a hydrogen bonding component (H), and a distance (Ra) of less than or equal to 13.4 MPa^{0.5} per the Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.8 MPa^{0.5},
P₁ is 4.8 MPa^{0.5}, and
H₁ is 13.0 MPa^{0.5}.

In a preferred embodiment, the one or more solvents have a dispersion component (D), a polar component (P), a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.4 MPa^{0.5}
P₁ is 5.0 MPa^{0.5}, and
H₁ is 11.7 MPa^{0.5}.

The solvent may be an oil. The term “oil” is intended to mean a non-aqueous compound, non-miscible in water, liquid at 25° C. and atmospheric pressure (760 mmHg; 1.013×10⁵ Pa). The solvent may be a non-silicone oil (e.g., an oil that does not contain silicon atoms, and in particular does not contain Si—O groups). Non-limiting examples of the solvents (b) include caprylic/capric triglyceride, isopropyl myristate, and polycitronellol acetate. The solvent may include acetone. The solvent may include oleic acid. The solvent may include an oleic acid containing oil (such as a vegetable oil). Table 1, below, shows values of D, P, and H, as well as Ra values for allowable ranges as well as preferred ranges, for several solvents.

TABLE 1

| | | | | Ra | Ra |
|---|---|---|---|---|---|
| | | | | (Allowable | (Preferred |
| Solvent (b) | D | P | H | Range) | Range) |
| Ethanol | 15.8 | 8.8 | 19.4 | 7.81 | 8.67 |
| Octyldodecanol | 16.1 | 2.2 | 7.4 | 6.33 | 5.17 |
| Isopropyl | 15.9 | 2.1 | 2.8 | 10.70 | 9.41 |
| Myristate | | | | | |
| Isopropyl Alcohol | 15.8 | 6.1 | 6.4 | 7.02 | 5.54 |
| Phenoxyethanol | 17.8 | 5.7 | 14.3 | 2.55 | 3.88 |
| CCTG | 18.22 | 5.39 | 14.74 | 3.38 | 4.76 |
| Castor Oil | 15.9 | 4.6 | 12 | 2.07 | 1.12 |
| Polycitronellol | 16.4 | 3 | 4.2 | 9.02 | 7.76 |
| Acetate | | | | | |
| Acetone | 15.5 | 10.4 | 7 | 8.61 | 7.38 |
| Oleic Acid | 16 | 2.8 | 6.2 | 7.27 | 5.98 |

In some embodiments, if oleic acid is utilized, at least some of the oleic acid may be provided by a vegetable oil. The vegetable oil may be a seed or nut oil. The vegetable oil may have an oleic acid content of at least 20% by weight of the vegetable oil. The vegetable oil may include sunflower oil, soybean oil, macadamia nut oil, and/or avocado oil. In some embodiments, the composition may include macadamia nut oil, and may be free, or substantially free, of other vegetable oils.

For purposes of the instant disclosure, the one or more of the solvents capable of solubilizing the hydrophobic polymer of (a) may not individually solubilize the hydrophobic polymer but when combined with other solvents, the combination solubilizes the hydrophobic polymer. Thus, when referring to a total amount of one or more solvents capable of solubilizing the hydrophobic polymer, the inclusion of all solvents that in combination solubilize the hydrophobic polymer is intended, even if one or more solvents in the combination do not individually solubilize the hydrophobic polymer.

Nonlimiting examples of solvents usual for solubilizing the hydrophobic polymer of (a), individually or in combination with other solvents, include polycitronellol acetate, caprylic/capric triglyceride, isododecane, isohexadecane, tetradecane, isopropyl myristate, octyldodecanol, ethanol, phenoxyethanol, castor oil, and mixtures thereof. In a preferred embodiment, at least one of the one or more solvents capable of solubilizing the hydrophobic polymer are selected from caprylic/capric triglyceride, polycitronellol acetate, isododecane, and mixtures thereof. In another preferred embodiment, at least one of the one or more solvents capable of solubilizing the hydrophobic polymer is polycitronellol acetate.

Nonlimiting solvent that individually or in combination with other solvents are useful for solubilizing the hydrophobic polymer of (a) include dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof.

Additionally useful solvents include hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesameseed oil, hazelnut oil, apricot oil, macadamia oil, arara oil, coriander oil, castor oil, avocado oil, caprylic/capric acid triglycerides, for instance those sold by the company Stearineries Dubois or those sold under the names MIGLYOL 810, 812 and 818 by the company Dynamit Nobel, jojoba oil, shea butter oil and caprylyl glycol; synthetic esters and ethers, especially of fatty acids, for instance Purcellin oil, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate, sold especially under the trade name ELDEW SL 205 by the company Ajinomoto; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam oil, or the mixture of n-undecane (C11) and of n-tridecane (C13) sold under the reference CETIOL UT by the company Cognis; fluoro oils that are partially hydrocarbon-based and/or silicone-based, for instance those described in document JP-A-2 295 912; silicone oils, for instance volatile or non-volatile polymethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes; mixtures thereof.

Additional specific examples solvents include cocoglyceride, cyclomethicone, dimethicone, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acids such as oleic acid, stearic acid, fatty alcohols such as cetyl alcohol, hexadecyl alcohol, diisopropyl adipate, hydroxybenzoate esters, benzoic acid esters of C9-C15 alcohols, isononyl iso-nonanoate, alkanes, mineral oil, silicone, dimethyl polysiloxane, ether, polyoxypropylene butyl ether, polyoxypropylene cetyl ether, C12-C15 alkyl benzoate, aryl alkyl benzoate, isopropyl lauroyl sarcosinate, or mixtures thereof.

The total amount of the one or more solvents capable of solubilizing the hydrophobic polymer of (a) in the mineral sunscreen composition will vary but can be from about 0.1 to about 60 wt. %, based on the total weight of the sunscreen composition. In certain embodiments, in can be preferably for the sunscreen composition to have a substantial oil phase. In such instances, the total amount of the one or more solvents can be from about 10 to about 60 wt. %, about 20 to about 60 wt. %, about 30 to about 60 wt. %, about 50 to about 60 wt. %, about 10 to about 40 wt. %, about 20 to about 40 wt. %, or about 25 to about 45 wt. %, based on the total weight of the sunscreen composition. In further embodiments, in can be preferably for the sunscreen composition to have a small oil phase. In such instances, the total amount of the one or more solvents can be from about 0.05 to about 10 wt. %, based on the total weight of the mineral sunscreen composition. In further embodiments, the total amount of the one or more solvents capable of solubilizing the hydrophobic polymer of (a) in the mineral sunscreen composition is from about 0.05 to about 8 wt. %, about 0.05 to about 5 wt. %, about 0.05 to about 3 wt. %, about 0.1 to about 10 wt. %, about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, or about 0.5 to about 3 wt. %, based on the total weight of the mineral sunscreen composition.

### (c) Mineral UV Filtering Agents

Nonlimiting examples of mineral UV filtering agent include treated or untreated metal oxides such as, for example, pigments or nanopigments of titanium oxide (amorphous or crystallized in rutile and/or anatase form), of iron oxide, of zinc oxide, of zirconium oxide or of cerium oxide. Particularly preferred mineral UV filtering agents include titanium dioxide and/or zinc oxide.

In some instances, the mean particle size of the mineral UV filtering agent may be about 5 nm to about 25 μm, about 10 nm to about 10 μm, or about 15 nm to about 5 μm. The mineral UV filtering agents may be nano-pigments having a mean particle size of about 5 nm to about 100 nm, about 5 nm to about 75 nm, or about 10 nm to 50 nm. Larger particles sizes may also be useful, for example about 1 μm to about 25 μm, about 5 μm to about 20 μm, or about 10 μm to about 15 μm.

Treated pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina or glycerol.

The treated pigments may be titanium oxides treated with:
silica and alumina, such as the products “MICROTITANIUM DIOXIDE MT 500 SA” and “MICROTITANIUM Dioxide MT 100 SA” from the company Tayca, and the products “TIOVEIL FIN”, “TIOVEIL OP”, “TIOVEIL MOTG” and “TIOVEIL IPM” from the company Tioxide;
alumina and aluminium stearate, such as the product “MICROTITANIUM DIOXIDE MT 100 T” from the company Tayca;
alumina and aluminium laurate, such as the product “MICROTITANIUM DIOXIDE MT 100 S” from the company Tayca;
iron oxides and iron stearate, such as the product “MICROTITANIUM DIOXIDE MT 100 F” from the company Tayca;
silica, alumina and silicone, such as the products “MICROTITANIUM DIOXIDE MT 100 SAS”, “Microtitanium Dioxide MT 600 SAS” and “MICROTITANIUM DIOXIDE MT 500 SAS” from the company Tayca;
sodium hexametaphosphate, such as the product “MICROTITANIUM DIOXIDE MT 150 W” from the company Tayca;
octyltrimethoxysilane, such as the product “T-805” from the company Degussa;
alumina and stearic acid, such as the product “UVT-M160” from the company Kemira;
alumina and glycerol, such as the product “UVT-M212” from the company Kemira;
alumina and silicone, such as the product “UVT-M262” from the company Kemira.

Other titanium oxide pigments treated with a silicone are TiO₂ treated with octyltrimethylsilane and for which the mean size of the elementary particles is between 25 and 40 nm, such as the product sold under the trade name “T805” by the company Degussa Silices, TiO₂ treated with a polydimethylsiloxane and for which the mean size of the elementary particles is 21 nm, such as the product sold under the trade name “70250 CARDRE UF TiO2SI3” by the company Cardre, anatase/rutile TiO₂ treated with a polydimethylhydrogenosiloxane and for which the mean size of the elementary particles is 25 nm, such as the product sold under the trade name “MICROTITANIUM DIOXIDE USP GRADE HYDROPHOBIC” by the company Color Techniques.

Uncoated titanium oxide pigments are sold, for example, by the company Tayca under the trade names “MICROTITANIUM DIOXIDE MT 500 B” or “MICROTITANIUM DIOXIDE MT 600 B”, by the company Degussa under the name “P 25”, by the company Wackher under the name “OXYDE DE TITANE TRANSPARENT PW”, by the company Myoshi Kasei under the name “UFTR”, by the company Tomen under the name “ITS” and by the company Tioxide under the name “TIOVEIL AQ”.

The uncoated zinc oxide pigments are, for example:
those sold under the name “Z-Cote” by the company Sunsmart;
those sold under the name “NANOX” by the company Elementis; and
those sold under the name “NANOGARD WCD 2025” by the company Nanophase Technologies.

The coated zinc oxide pigments are, for example:
those sold under the name “ZINC OXIDE CS-5” by the company Toshibi (ZnO coated with polymethylhydrogenosiloxane);
those sold under the name “NANOGARD ZINC OXIDE FN” by the company Nanophase Technologies (as a 40% dispersion in FINSOLV TN, C12-C15 alkyl benzoate);
those sold under the name “DAITOPERSION ZN-30” and “DAITOPERSION ZN-50” by the company Daito (dispersions in cyclopolymethylsiloxane/oxyethylenated polydimethylsiloxane, containing 30% or 50% of nanozinc oxides coated with silica and polymethylhydrogenosiloxane);
those sold under the name “NFD ULTRAFINE ZNO” by the company Daikin (ZnO coated with perfluoroalkyl phosphate and copolymer based on perfluoroalkylethyl as a dispersion in cyclopentasiloxane);
those sold under the name “SPD-Z1” by the company Shin-Etsu (ZnO coated with silicone-grafted acrylic polymer, dispersed in cyclodimethylsiloxane);
those sold under the name “ESCALOL Z100” by the company ISP (alumina-treated ZnO dispersed in an ethylhexyl methoxycinnamate/PVP-hexadecene/methicone copolymer mixture);
those sold under the name “FUJI ZNO-SMS-10” by the company Fuji Pigment (ZnO coated with silica and polymethylsilsesquioxane); and
those sold under the name “NANOX Gel TN” by the company ELEMENTIS (ZnO dispersed at a concentration of 55% in C₁₂-C₁₅ alkyl benzoate with hydroxystearic acid polycondensate).

The uncoated cerium oxide pigments are sold under the name “COLLOIDAL CERIUM OXIDE” by the company Rhone-Poulenc. The uncoated iron oxide nanopigments are sold, for example, by the company Arnaud under the names “NANOGARD WCD 2002 (FE 45B)”, “NANOGARD Iron FE 45 BL AQ”, “NANOGARD FE 45R AQ” and “NANOGARD WCD 2006 (FE 45R)” or by the company Mitsubishi under the name “TY-220”. The coated iron oxide nanopigments are sold, for example, by the company Arnaud under the names “NANOGARD WCD 2008 (FE 45B FN)”, “NANOGARD WCD 2009 (FE 45B 556)”, “NANOGARD FE 45 BL 345” and “Nanogard FE 45 BL” or by the company BASF under the name “TRANSPARENT IRON OXIDE”.

Mixtures of metal oxides may also be used, especially of titanium dioxide and of cerium dioxide, including the silica-coated equal-weight mixture of titanium dioxide and of cerium dioxide, sold by the company Ikeda under the name “SUNVEIL A”, and also the alumina, silica and silicone-coated mixture of titanium dioxide and of zinc dioxide, such as the product “M 261” sold by the company Kemira, or the alumina, silica and glycerol-coated mixture of titanium dioxide and of zinc dioxide, such as the product “M 211” sold by the company Kemira.

The total amount of mineral UV filtering agents in the mineral sunscreen compositions can vary but is typically about 1 to about 30 wt. %, based on the total weight of the mineral sunscreen composition. In some instances, the total amount of mineral UV filtering agents may be about 1 to about 25 wt. %, about 1 to about 20 wt. %, about 1 to about 15 wt. %, about 1 to about 10 wt. %, about 5 to about 30 wt. %, about 5 to about 25 wt. %, about 5 to about 20 wt. %, about 5 to about 5 wt. %, about 5 to about 10 wt. %, based on the total weight of the mineral sunscreen composition.

### (d) Surfactant

For purposes of the instant disclosure, the term “surfactant” includes emulsifiers and detergents. Surfactants, or surface-active agents, are compounds that lower the surface tension between two liquids or between a liquid and a solid. Surfactants are amphiphilic, meaning that they contain hydrophilic (water-loving) head groups and hydrophobic (water-hating, or oil-loving) tails. Surfactants adsorb at the interface between oil and water, thereby decreasing the surface tension.

For purposes of the instant disclosure, an “emulsifier” is a surfactant that stabilizes emulsions. Emulsifiers coat droplets within an emulsion and prevent them from coming together, or coalescing. An “emulsion” is a mixture of two or more liquids, with or without an emulsifier, that are normally immiscible. One of the liquids, the “dispersed phase,” forms droplets in the other liquid, the “continuous phase.”

A “detergent” is a surfactant that has cleaning properties in dilute solutions and is typically anionic.

The surfactants can be anionic, cationic, amphoteric (zwitterionic), or nonionic. Preferably, the emulsions of the instant case include one or more surfactants selected from anionic surfactants, amphoteric (zwitterionic) surfactants, nonionic surfactants, or mixtures thereof. In various embodiments, the emulsions are preferably free or essentially free from cationic surfactants. In other embodiments, the emulsions include one or more cationic surfactants. In preferred embodiments, the emulsions contain one or more biosurfactants, one or more anionic surfactants, optionally, one or more nonionic surfactants, or mixtures thereof.

In a preferred embodiment, the compositions of the instant disclosure include a plurality of surfactants, wherein the plurality of surfactants include one or more biosurfactants and one or more surfactants other than the one or more biosurfactants. In further embodiments, the compositions of the instant disclosure include one or more biosurfactants, one or more anionic surfactants, and optionally, one or more nonionic surfactants.

In a preferred embodiment, the mineral sunscreen composition includes one or more biosurfactants. In further embodiments, the mineral sunscreen composition includes one or more biosurfactants and one or more additional surfactants different from the one or more biosurfactants selected from anionic surfactants, nonionic surfactants, amphoteric surfactants, or combinations thereof.

### Biosurfactant

Biosurfactants are amphiphilic molecules, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes.

Biosurfactants are environmentally friendly, biodegradable, and non-toxic and may be classified into high and low molecular weight biosurfactants. Low molecular weight biosurfactant efficiently lower surface and interfacial tension, and high molecular weight biosurfactants are more effective as emulsion-stabilizing agents. Examples of low molecular weight biosurfactants include glycolipids, such as rhamnolipids, sophorolipids, lipopeptidesm, and trehalolipids. These low molecular weight biosurfactants have hydrophilic heads comprised of sugar units linked glycosidically with hydrophobic non-polar parts. Examples of high molecular weight biosurfactants include polysaccharides, lipopolysaccharides, proteins, and lipoproteins. Polysaccharide-based biosurfactant can be classified into sorbitan esters, sucrose esters and glucose-based surfactants that include alkyl polyglycosides and fatty acid glucamides.

Nonlimiting examples of biosurfactants include liptopeptides such as surfactin; fatty acids and phospholipids, polymeric matrix biosurfactants; particulate biosurfactants; and bacterial biosurfactants composed of polysaccharides, proteins, lipopolysaccharides, lipoproteins or complex mixtures of these biopolymers.

Nonlimiting examples of commercially available biosurfactants includealkyl polyglycoside available under the trademark ECOSENSE® 3000 from Dow Chemical®; D-glucopyranose, oligomeric, decyl octyl glycosides available under the trademark GLUCOPON® 215 from BASF Corporation®; rhamnolipids available under the trademark REWOFERM® SL ONE from Evonik®; D-Glucitol, 1-deoxy-1-(methylamino)-, N-coco acyl derivatives available under the trademark GLUCOTAIN® from Clariant®; rhamnolipids from Jeneil Biotech®, and BioLoop® surfactants from Lankem® Ltd.

In one embodiment, the microbial biosurfactant is a glycolipid such as rhamnolipids (RLP), sophorolipids (SLP), trehalose lipid or mannosylerythritol lipid (MEL). The biosurfactants can be added in purified form or can be present in the microbe-based composition as a result of microbial growth. The biosurfactant may be a sophorolipid. In some embodiments, the biosurfactant can also be a lipopeptide, such as surfactin, and/or a rhamnolipid.

In some embodiments, a blend of biosurfactants is present. Preferably the blend comprises a rhamnolipid, and optionally one or both of a mannosylerythritol lipid, a surfactin or a sophorolipid. In a preferred embodiment, the microbe is a non-pathogenic strain of Pseudomonas. Preferably, the strain is a producer of rhamnolipid (RLP) biosurfactants.

Other microbial strains including, for example, other fungal strains capable of accumulating significant amounts of, for example, glycolipid-biosurfactants can be used in accordance with the subject invention. Biosurfactants useful according to the present invention include mannoprotein, beta-glucan and other metabolites that have bio-emulsifying and surface/interfacial tension-reducing properties.

In various embodiments, the one or more biosurfactants are selected from surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, and combinations thereof. In one embodiment, the biosurfactants are selected from one or more glycolipids such as, for example, rhamnolipids, rhamnose-d-phospholipids, trehalose lipids, trehalose dimycolates, trehalose monomycolates, mannosylerythritol lipids, cellobiose lipids, ustilagic acid and/or sophorolipids.

In various embodiments, the biosurfactant has an anionic character, for example, sophorolipids, trehalolipid and rhamnolipids. Preferable are the mono-rhamnolipids and di-rhamnolipids. The preferred alkyl chain length is from C to C12. The alkyl chain may be saturated or unsaturated.

The term “rhamnolipids” includes compounds of the general formula (II) and salts thereof,

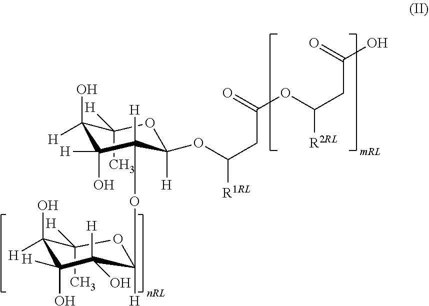

wherein,
mRL=2, 1 or 0,
nRL=1 or 0,
R^{1RL} and R^{2RL}=are independently organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CHs where o=1 to 23, preferably 4 to 12.

If nRL=1, the glycosidic bond between the two rhamnose units is preferably in the α-configuration. The optically active carbon atoms of the fatty acids are preferably present as R-enantiomers (e.g. I-3-{I-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (II) or salts thereof, where nRL=1.

The term “mono-rhamnolipid” in the context of the present invention is understood to mean compounds of the general formula (II) or salts thereof, where nRL=0.

Distinct rhamnolipids are abbreviated according to the following nomenclature: “diRL-CXCY” are understood to mean di-rhamnolipids of the general formula (II), in which one of the residues R^{1RL} and R^{2RL}=(CH2)ₒ—CH₃ where o=X-4 and the remaining residue R¹ or R²—(CH₂)ₒ—CH₃ where o=Y-4.

“monoRL-CXCY” are understood to mean mono-rhamnolipids of the general formula (II), in which one of the residues R^{1RL} and R^{2RL}═(CH.sub.₂).sub.ₒ—CH.sub.₃ where o=X-4 and the remaining residue R^{1RL} or R^{2RL}═(CH2)ₒ—CH₃ where o=Y-4. The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “: Z”, this signifies that the respective residue R^{1RL} and/or R^{2RL} is equal to an unbranched, unsubstituted hydrocarbon residue having X-3 or Y-3 carbon atoms having Z double bonds.

Methods for preparing the relevant rhamnolipids are disclosed, for example, in EP2786743 and EP2787065, which are incorporated herein by reference in their entirety. Rhamolipids can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been Improved for higher rhamnolipid titres by genetical modification can also be used in the context of the instant invention; such cells have for example been disclosed by Lei et al. in BIOTECHNOL LETT. 2020 June; 42 (6): 997-1002, which is incorporated herein by reference in its entirety. The biosurfactants, in particular glycolipid surfactants, can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789, which are incorporated herein by reference in their entirety.

Rhamnolipids produced by Pseudomonas aeruginosa are commercially available from Jeneil Biotech Inc., e.g. under the tradename ZONIX®, from Logos Technologies (technology acquired by Stepan), e.g. under the tradename NATSURFACT®, from Biotensidon GmbH, e.g. under the tradename RHAPYNAL®, from AGAE® technologies, e.g. under the name R90, R95, R95Md, R95Dd, from Locus Bio-Energy Solutions and from Shanghai Yusheng Industry Co. Ltd., e.g. under the tradename BIO-201 GLYCOLIPIDS®.

The total amount of the one or more biosurfactants in the mineral sunscreen composition, if present, will vary but may be from about 0.1 to about 15 wt. %, based on the total weight of the mineral sunscreen composition. In further embodiments, the total amount of the one or more biosurfactants in the mineral sunscreen composition is from about 0.1 to about 10 wt. %, or about 0.1 to about 5 wt. %. In a further embodiment, the total amount of the one or more biosurfactants in the mineral sunscreen composition is from about 0.5 to about 15 wt. %, about 0.5 to about 10 wt. %, or about 0.5 to about 5 wt. %, based on the total weight of the mineral sunscreen composition. In yet a further embodiment, the total amount of the one or more biosurfactants in the mineral sunscreen composition is from about 1 to about 15 wt. %, about 1 to about 10 wt. %, or about 1 to about 5 wt. %, based on the total weight of the mineral sunscreen composition. In preferred embodiments, the total amount of the one or more biosurfactants in the mineral sunscreen composition is from about 1 to about 8 wt. %, about 1 to about 6 wt. %, about 1 to about 5 wt. %, about 1 to about 4 wt. %, or about 1 to about 3 wt. %, based on the total weight of the mineral sunscreen composition.

### Anionic Surfactants

Popular anionic surfactants known for their good detersive and foaming properties include sodium lauryl sulfate and sodium laureth ether sulfate. Sulfate-based surfactants may optionally be included. In other embodiments, it is preferable that the mineral sunscreen composition is free or essentially free from sulfate-based surfactants. In some instances, it can be useful to include a gentler, non-sulfate anionic surfactant, such as one or more anionic surfactants selected from alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof.

### (a) Acyl Isethionates

Non-limiting examples of useful acyl isethionates include those of formula (III) and (IV):

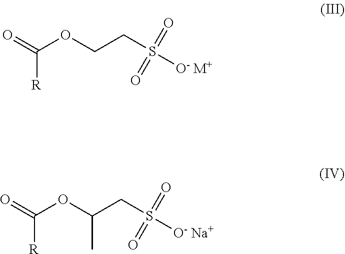

wherein R, R¹, R² and R³ are each independently selected from H or an alkyl chain having 1-24 carbon atoms, said chain being saturated or unsaturated, linear or branched, and X is COO⁻ or SO₃⁻. Although sodium is shown as the cation in formulae (III) and (IV), the cation for both formula (III) and formula (IV) may be an alkali metal ion such as sodium or potassium, ammonium ions, or alkanolammonium ions such as monoethanolammonium or triethanolammonium ions. Non-limiting examples of acyl isethionates include sodium isethionate, sodium cocoyl isethionate, sodium lauroyl methyl isethionate, and sodium cocoyl methyl isethionate. In some embodiments, a combination of sodium isethionate and sodium cocoyl isethionate are preferable.

### (b) Alkyl Sulfonates

Examples of alkyl sulfonates include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful.

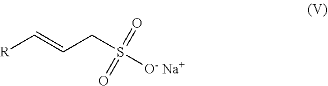

R is selected from H or alkyl chain that has 1-24 carbon atoms, preferably 6-24 carbon atoms, more preferably, 8 to 20 carbon atoms, said chain being saturated or unsaturated, linear or branched. Sodium is shown as the cation in the above formula (V) but the cation may be an alkali metal ion such as sodium or potassium, ammonium ions, or alkanolammonium ions such as monoethanolammonium or triethanolammonium ions. In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, and mixtures thereof. C₁₀-C₂₄ olefin sulfonates may be particularly preferred. A non-limiting example of a C₁₀-C₂₄ olefin sulfonate that can be used in the instant compositions is sodium C₁₄-C₁₆ olefin sulfonate.

### (c) Alkyl Sulfosuccinates

Non-limiting examples of useful sulfosuccinates include those of formula (VI):

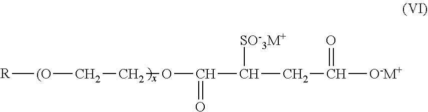

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

### (d) Alkyl Sulfoacetates

Non-limiting examples of alkyl sulfacetates includes, for example, alkyl sulfoacetates such as C4-C18 fatty alcohol sulfoacetates and/or salts thereof. A particularly preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

### (e) Alkoxylated Monoacids

Non-limiting examples of alkoxylated monoacids include compounds corresponding to formula (VII):

RO[CH2O]u[(CH2)xCH(R′)(CH2)y(CH2)zO]v[CH2CH2O]wCH2COOH (VII)
wherein:
R is a hydrocarbon radical containing from about 6 to about 40 carbon atoms;
u, v and w, independently of one another, represent numbers of from 0 to 60;
x, y and z, independently of one another, represent numbers of from 0 to 13;
R′ represents hydrogen, alkyl, and
the sum of x+y+z>0;

Compounds corresponding to formula (VII) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C₆-C₄₀ alkyl or alkenyl group or a C₁-C₄₀ alkyl phenyl group, more typically a C₈-C₂₂ alkyl or alkenyl group or a C₄-C₁₈ alkyl phenyl group, and even more typically a C₁₂-C₁₈ alkyl group or alkenyl group or a C₆-C₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C₉-C₁₁ Pareth-6 Carboxylic Acid, C₁₁-C₁₅ Pareth-7 Carboxylic Acid, C₁₂-C₁₃ Pareth-5 Carboxylic Acid, C₁₂-C₁₃ Pareth-8 Carboxylic Acid, C₁₂-C₁₃ Pareth-12 Carboxylic Acid, C₁₂-C₁₅ Pareth-7 Carboxylic Acid, C₁₂-C₁₅ Pareth-8 Carboxylic Acid, C₁₄-C₁₅ Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and a mixture thereof.

### (f) Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of acyl amino acids include those of formula (VIII):

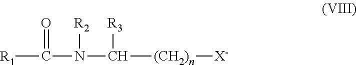

wherein R, R¹, R² and R³ are each independently selected from H or an alkyl chain having 1-24 carbon atoms, said chain being saturated or unsaturated, linear or branched, and X is COO⁻ or SO₃⁻.

### (g) Acyl Taurates

Non-limiting examples of acyl taurates include those of formula (IX):

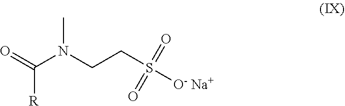

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate, sodium methyl cocoyl taurate, sodium lauroyl taurate, and sodium methyl lauroyl taurate.

### (h) Acyl Glycinates

Non-limiting examples of acyl glycinates include those of formula (X):

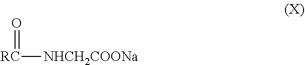

wherein R is an alkyl chain of 8 to 16 carbon atoms. Although sodium is shown as the cation in the above formula (X), the cation may be an alkali metal ion such as sodium or potassium, ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

### (i) Acyl Glutamates

Non-limiting examples of acyl glutamates include those of formula (XI):

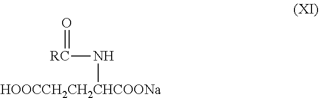

wherein R is an alkyl chain of 8 to 16 carbon atoms. Sodium is shown as the cation in the above formula (XI) but the cation may be an alkali metal ion such as sodium or potassium, ammonium ions, or alkanolammonium ions such as monoethanolammonium or triethanolammonium ions. Non-limiting examples of acyl gluatamtes include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

### (j) Acyl Sarcosinates:

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

The total amount of the one or more anionic surfactants in the mineral sunscreen composition, if present, will vary but may be from about 0.01 to about 6 wt. %, based on the total weight of the mineral sunscreen composition. In further embodiments, the total amount of the one or more anionic surfactants in the mineral sunscreen composition, if present, is from about 0.01 to about 5 wt. %, about 0.01 to about 3 wt. %, about 0.1 to about 6 wt. %, about 0.1 to about 5 wt. %, about 0.1 to about 3 wt. %, about 0.5 to about 6 wt. %, about 0.5 to about 5 wt. %, about 0.5 to about 3 wt. %, based on the total weight of the mineral sunscreen composition.

### Amphoteric Surfactants

The multiphase cleansing compositions may optionally include one or more amphoteric surfactants. Nonlimiting examples of amphoteric surfactants include betaines, alkyl amphoacetates and alkyl amphodiacetates, alkyl sulltaines, alkyl amphopropionates, and combinations thereof.

Nonlimiting examples of betaine surfactants include coco-betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, or mixtures thereof.

Nonlimiting examples of alkyl amphoacetates and alkyl amphodiacetates include (C8-C20)alkylamphoacetates and (C8-C20)alkylamphodiacetates, such as disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylampho-dipropionate, disodium caprylomphodipropionate, lauroamphodipropionic acid, cocoamphodipropionic acid, and combinations thereof.

The total amount of the one or more amphoteric surfactants in the mineral sunscreen composition, if present, will vary but may be from about 0.01 to about 6 wt. %, based on the total weight of the mineral sunscreen composition. In further embodiments, the total amount of the one or more amphoteric surfactants in the mineral sunscreen composition, if present, is from about 0.01 to about 5 wt. %, about 0.01 to about 3 wt. %, about 0.1 to about 6 wt. %, about 0.1 to about 5 wt. %, about 0.1 to about 3 wt. %, about 0.5 to about 6 wt. %, about 0.5 to about 5 wt. %, about 0.5 to about 3 wt. %, based on the total weight of the mineral sunscreen composition.

### Nonionic Surfactants

The multiphase cleansing composition may optionally include one or more nonionic surfactants. Nonlimiting examples of nonionic surfactants include alkoxylated fatty alcohols, alkoxylated polyol esters, alkoxylated glycerides, glucosides, alkanolamides, sorbitan derivatives, or combinations thereof.

Nonlimiting examples of alkoxylated fatty alcohols include laureth-3, laureth-4, laureth-7, laureth-9, laureth-12, laureth-23, ceteth-10, steareth-10, steareth-2, steareth-100, beheneth-5, beheneth-5, beheneth-10, oleth-10, Pareth alcohols, trideceth-10, trideceth-12, C12-13 pareth-3, C12-13 pareth-23, C11-15 pareth-7, PEG hydrogenated castore oil, PEG-75 lanolin, polysorbate-80, polysobate-20, PPG-5 ceteth-20, PEG-55 Propylene Glycol Oleate, glycereth-26 (PEG-26 Glyceryl Ether), PEG 120 methyl glucose dioleate, PEG 120 methyl glucose trioleate, PEG 150 pentaerythrityl tetrastearate, and mixtures thereof.

Nonlimiting examples of alkoxylatecd polyol esters include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and mixtures thereof.

Nonlimiting examples of alkoxylated glycerides include PEG-6 almond glycerides, PEG-20 almond glycerides, PEG-35 almond glycerides, PEG-60 almond glycerides, PEG-192 apricot kernel glycerides, PEG-11 avocado glycerides, PEG-14 avocado glycerides, PEG-11 babassu glycerides, PEG-42 babassu glycerides, PEG-4 caprylic/capric glycerides, PEG-6 caprylic/capric glycerides, PEG-7 caprylic/capric glycerides, PEG-8 caprylic/capric glycerides, PEG-11 cocoa butter glycerides, PEG-75 cocoa butter glycerides, PEG-7 cocoglycerides, PEG-9 cocoglycerides, PEG-20 corn glycerides, PEG-60 corn glycerides, PEG-20 evening primrose glycerides, PEG-60 evening primrose glycerides, PEG-5 hydrogenated corn glycerides, PEG-8 hydrogenated fish glycerides, PEG-20 hydrogenated palm glycerides, PEG-6 hydrogenated palm/palm kernel glyceride, PEG-16 macadamia glycerides, PEG-70 mango glycerides, PEG-13 mink glycerides, PEG-25 moringa glycerides, PEG-42 mushroom glycerides, PEG-2 olive glycerides, PEG-6 olive glycerides, PEG-7 olive glycerides, PEG-10 olive glycerides, PEG-40 olive glycerides, PEG-18 palm glycerides, PEG-12 palm kernel glycerides, PEG-45 palm kernel glycerides, PEG-60 Passiflora edulis seed glycerides, PEG-60 Passiflora incarnata seed glycerides, PEG-45 safflower glycerides, PEG-60 shea butter glycerides, PEG-75 shea butter glycerides, PEG-75 shorea butter glycerides, PEG-35 soy glycerides, PEG-75 soy glycerides, PEG-2 sunflower glycerides, PEG-7 sunflower glycerides, PEG-10 sunflower glycerides, PEG-13 sunflower glycerides, PEG-5 tsubakiate glycerides, PEG-10 tsubakiate glycerides, PEG-20 tsubakiate glycerides, PEG-60 tsubakiate glycerides, sodium PEG-8 palm glycerides carboxylate, or mixtures thereof.

Nonlimiting examples of glucosides (also known as “alkyl polyglucosides”) include lauryl glucoside, octyl glucoside, decyl glucoside, coco glucoside, caprylyl/capryl glucoside, sodium lauryl glucose carboxylate, or combinations thereof.

Nonlimiting examples of alkanolamides include cocamide MIPA, cocamide DEA, cocamide MEA, cocamide DIPA, and mixtures thereof.

Nonlimiting examples of sorbitan derivatives include polysorbates, for example, polysorbate-20 (POE(20) sorbitan monolaurate), polysorbate-21 (POE(4) sorbitan monolaurate), polysorbate-40 (POE(20) sorbitan monopalmitate), polysorbate-60 (POE(20) sorbitan monostearate), polysorbate-61 (POE(4) sorbitan monostearate), polysorbate-65 (POE(20) sorbitan tristearate), polysorbate-80 (POE(20) sorbitan monooleate), polysorbate-81 (POE(4) sorbitan monooleate), polysorbate 85 (POE(20) Sorbitan Trioleate), sorbitan isostearate, sorbitan monolaurate, sorbitan monooleate, sorbitan monopalmitate, sorbitan monostearate, sorbitan sesquioleate, sorbitan trioleate sorbitan tristearate, or mixtures thereof.

The total amount of the one or more nonionic surfactants in the mineral sunscreen composition, if present, will vary but may be from about 0.01 to about 6 wt. %, based on the total weight of the mineral sunscreen composition. In further embodiments, the total amount of the one or more nonionic surfactants in the mineral sunscreen composition, if present, is from about 0.01 to about 5 wt. %, about 0.01 to about 3 wt. %, about 0.1 to about 6 wt. %, about 0.1 to about 5 wt. %, about 0.1 to about 3 wt. %, about 0.5 to about 6 wt. %, about 0.5 to about 5 wt. %, about 0.5 to about 3 wt. %, based on the total weight of the mineral sunscreen composition.

### Cationic Surfactants

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, and mixtures thereof.

The one or more cationic surfactants may be selected from quaternary ammonium compounds, fatty dialkylamines, or mixtures thereof.

Nonlimiting examples of quaternary ammonium compounds include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and combinations thereof.

Nonlimiting examples of fatty dialkylamines include oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, and combinations thereof.

In various embodiments, the one or more cationic surfactants are preferably selected from cetrimonium chloride, behentrimonium chloride, behentrimonium methosulfate, stearamidopropyl dimethylamine, brassicamidopropyl dimethylamine or a mixture thereof.

The total amount of the one or more cationic surfactants in the mineral sunscreen composition, if present, will vary but may be from about 0.01 to about 6 wt. %, based on the total weight of the mineral sunscreen composition. In further embodiments, the total amount of the one or more cationic surfactants in the mineral sunscreen composition, if present, is from about 0.01 to about 5 wt. %, about 0.01 to about 3 wt. %, about 0.1 to about 6 wt. %, about 0.1 to about 5 wt. %, about 0.1 to about 3 wt. %, about 0.5 to about 6 wt. %, about 0.5 to about 5 wt. %, about 0.5 to about 3 wt. %, based on the total weight of the mineral sunscreen composition.

### (d) Water

The total amount of water in the mineral sunscreen composition will vary but may be from about 30 to about 90 wt. %, based on the total weight of the mineral sunscreen composition. In further embodiments, the total amount of water may be from about 30 to about 80 wt. %, about 30 to about 70 wt. %, about 30 to about 60 wt. %, about 30 to about 50 wt. %, about 40 to about 90 wt. %, about 40 to about 80 wt. %, about 40 to about 70 wt. %, about 40 to about 60 wt. %, about 40 to about 50 wt. %, about 50 to about 90 wt. %, about 50 to about 80 wt. %, about 50 to about 70 wt. %, about 50 to about 60 wt. %, about 60 to about 90 wt. %, about 60 to about 80 wt. %, about 50 to about 70 wt. %, based on the total weight of the mineral sunscreen composition.

### Organic UV Filtering Agents

The mineral sunscreen compositions may optionally include one or more organic UV filtering agents in addition to one or more mineral organic filtering agents. In certain embodiments, the mineral sunscreen compositions are free or essentially free from organic UV filtering agents.

Organic UV filters are known in the art for their use in stopping UV radiation. Nonlimiting examples of organic UV filtering agents include:
i. Sparingly soluble UV filters (not appreciably soluble in either water or oil) such as Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phen-yl]- and mixtures thereof.
ii. Oil soluble organic UV filters (at least partially soluble in oil or organic solvent), such as Bis-Ethylhexyloxyphenol Methoxyphenyl Triazine, Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, and Diethylamino Hydroxy Benzoyl Hexyl Benzoate (DHHB);
iii. Water soluble UV filters such as Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, and PEG-25 PABA.

In some instances, one or more organic UV filtering agents is a para-aminobenzoic acid derivative, a salicylic derivative, a cinnamic derivative, a benzophenone or an aminobenzophenone, an anthranilic derivative, a β,β-diphenylacrylate derivative, a benzylidenecamphor derivative, a phenylbenzimidazole derivative, a benzotriazole derivative, a triazine derivative, a bisresorcinyl triazine, an imidazoline derivative, a benzalmalonate derivative, a 4,4-diarylbutadiene derivative, a benzoxazole derivative, a merocyanine, malonitrile or a malonate diphenyl butadiene derivative, a chalcone, or a mixture thereof.

Furthermore, combinations of UV filtering agents may be used. For example, a combination of UV filters may include one or more of octocrylene, avobenzone (butyl methoxydibenzoylmethane), oxybenzone (benzophenone-3), octisalate (ethylhexyl salicylate), and homosalate. In another embodiment, at least one mineral UV filtering agent is combined at least one organic UV filtering agent, for example, one or more organic UV filtering agent selected from octocrylene, avobenzone, octisalate, homosalate, oxybenzone, or combinations thereof. In another embodiment, at least one mineral UV filtering agents is combined with one or more organic UV filtering agents, for example, one or more organic UV filtering agents selected from octocrylene, butyl methoxydibenzoylmethane, bis-ethylhexyloxyphenol methoxyphenyl triazine, ethylhexyl triazone, terephthalylidene dicamphor sulfonic acid, drometrizole trisiloxane, or mixtures thereof.

The total amount of the one or more organic UV filtering agents, if present, will vary but may be from about 0.1 to about 15 wt. %, based on the total weight of the mineral sunscreen composition. In further embodiments, the mineral sunscreen composition includes from about 0.1 to about 10 wt. %, about 0.1 to about 5 wt. %, about 0.5 to about 15 wt. %, about 0.5 to about 10 wt. %, about 0.5 to about 5 wt. %, based on the weight of the mineral sunscreen compositions.

### Film-Forming Polymers

The mineral sunscreen composition may optionally include one or more film-forming polymers, which are also referred to as simply “film formers.” The film forming polymer is preferably a hydrophobic material that imparts film forming and/or waterproofing characteristics to the mineral sunscreen composition upon application to the skin. One such agent is polyethylene, which is available from New Phase Technologies as Performalene® 400, a polyethylene having a molecular weight of 400. Another suitable film former is polyethylene 2000 (molecular weight of 2000), which is available from New Phase Technologies as Performalene®. Yet, another suitable film former is synthetic wax, also available from New Phase Technologies as Performa® V-825. Other typical film-formers include acrylates/acrylamide copolymer, acrylates copolymer, acrylates/C₁₂-C₂₂ alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, and mixtures thereof. In some cases, the film former is acrylates/C₁₂-C₂₂ alkylmethacrylate copolymer sold under the tradename Allianz OPT® by ISP.

Many of the common film-forming polymers included in sunscreen compositions are not soluble in ethanol (such as PVP/Eicosene copolymer). A common film-former employed in ethanol based sunscreen products is Dermacryl LT or Dermacryl 79 marketed by Akzo Nobel (INCI Name: acrylates/octylacrylamide copolymner). Dermacryl LT (CAS Number: 80570-62-3) is a hydrophobic, high molecular weight carboxylated acrylic copolymer. It functions as a film-former in a broad range of cosmetic formulations, imparting waterproofing, increased occlusivity and decreased rub-off of actives.

The one or more film-forming polymers may be, for example, sodium silicate, colloidal silica, pullulan, polycacrylate-21 (and) acrylates/dimethylamino-ethylmethacrylate copolymer, polyurethanes, polysaccharides, polyvinylpyrrolidone, polyacrylates, acrylate copolymers, and the mixtures thereof. In some cases, the mineral sunscreen composition includes at least one polysaccharide, which may have one or more free hydroxyl groups. Furthermore, in some cases, the polysaccharide is pullulan. In some cases, mineral sunscreen compositions include at least two film forming polymers, which may be any two film forming polymers.

Non-limiting examples of useful film forming polymers include acrylic acid, crotonic acid, methacrylic acid, maleic acid, itaconic acid and combinations and mixtures thereof. Additional film forming polymers, either synthetic or natural can be used with the acid containing polymers described above. Non-limiting examples of these additional film forming polymers are: from National Starch and Chemical Company, AMPHOMER®. and AMPHOMER® LV-71 polymers (octylacrylamide/acrylates/butylaminoethyl methacrylate copolymer), AMPHOMER® HC polymer (acrylates/octylacrylamide copolymer) BALANCE® 0/55 and BALANCE® CR polymers (acrylates copolymer), BALANCE® 47 polymer (octylacrylamide/butylaminoethyl methacrylate copolymer), RESYN® 28-2930 polymer (VA/crotonates/vinyl neodecanoate copolymer), RESYN® 28-1310 polymer (VA/Crotonates copolymer), FLEXAN® polymers (sodium polystyrene sulfonate), DynamX polymer (polyurethane-14 (and) AMP-Acrylates copolymer), RESYN® XP polymer (acrylates/octylacrylamide copolymer), STRUCTURE® 2001 (acrylates/steareth-20 itaconate copolymer) and STRUCTURE® 3001 (acrylates/ceteth-20 itaconate copolymer); from ISP, OMNIREZ-2000® (PVM/MA half ethyl ester copolymer), GANEX P-904® (butylated PVP), GANEX V-216® (PVP/hexadecene copolymer) GANEX® V-220 (PVP/eicosene copolymer), GANEX® WP-660 (tricontanyl PVP), GANTREZ® A425 (butyl ester of PVM/MA copolymer), GANTREZ® AN-119 PVM/MA copolymer, GANTREZ® ES 225 (ethyl ester of PVM/MA copolymer), GANTREZ® ES425 (butyl ester of PVM/MA copolymer), GAFFIX® VC-713 (vinyl caprolactam/PVP/dimethylaminoethyl methacrylate copolymer), GAFQUAT® 755 (polyquaternium-11), GAFQUAT HS-100® (polyquaternium-28) AQUAFLEX® XL-30 (Polyimide-1), AQUAFLEX® SP-40 (PVP/Vinylcaprolactam/DMAPA Acrylates Copolymer), AQUAFLEX® PX-64 (Isobutylene/Ethylmaleimide/Hydroxethylmaleimide Copolymer), ALLIANZ® LT-120 (Acrylates/C1-2 Succinates/Hydroxyacrylates Copolymer), STYLEZE® CC-10 (PVP/DMAPA Acrylates Copolymer), STYLEZE® 2000 (VP/Acrylates/Lauryl Methacrylate Copolymer), STYLEZE® W-20 (Polyquaternium-55), Copolymer Series (PVP/Dimethylaminoethylmethacrylate Copolymer), ADVANTAGE® S and ADVANTAGE® LCA (VinylcaprolactamNP/Dimethylaminoethyl Methacrylate Copolymer), ADVANTAGE® PLUS (VA/Butyl Maleate/Isobornyl Acrylate Copolymer); from BASF, ULTRAHOLD STRONG (acrylic acid/ethyl acrylate/t-butyl acrylamide), LUVIMER® 100P (t-butyl acrylate/ethyl acrylate/methacrylic acid), LUVIMER® 36D (ethyl acrylate/t-butyl acrylate/methacrylic acid), LUVIQUAT® HM-552 (polyquaternium-16), LUVIQUAT® HOLD (polyquaternium-16); LUVISKOL® K30 (PVP) LUVISKOL® K90 (PVP), LUVISKOL® VA 64 (PVP/VA copolymer) LUVISKOL® VA73W (PVP/VA copolymer), LUVISKOL® VA, LUVISET® PUR (Polyurethane-1), LUVISET® Clear (VP/MethacrylamideNinyl Imidazole Copolymer), LUVIFLEX® SOFT (Acrylates Copolymer), ULTRAHOLD® 8 (Acrylates/Acrylamide Copolymer), LUVISKOL® Plus (Polyvinylcaprolactam), LUVIFLEX® Silk (PEG/PPG-25/25 Dimethicone/Acrylates Copolymer); From Amerchol, AMERHOLD® DR-25 (acrylic acid/methacrylic acidlacrylates/methacrylates); from Rohm & Haas, ACUDYNE® 258 (acrylic acid/methacrylic acid/acrylates/methacrylates/hydroxy ester acrylates; from Mitsubishi and distributed by Clariant, DIAFORMER® Z-301, DIAFORMER® Z-SM, and DIAFORMER® Z-400 (methacryloyl ethyl betaine/acrylates copolymer), ACUDYNE® 180 (Acrylates/Hydroxyesters Acrylates Copolymer), ACUDYNE® SCP (Ethylenecarboxyamide/AMPSA/Methacrylates Copolymer), and the ACCLTLYN® rheological modifiers; from ONDEO Nalco, FIXOMER® A-30 and FIXOMER® N-28 (INCI names: methacrylic acid/sodium acrylamidomethyl propane sulfonate copolymer); from Noveon, FIXATE® G-100 (AMP-Acrylates/Allyl Methacrylate Copolymer), FIXATE PLUS® (Polyacrylates-X), CARBOPOL® Ultrez 10 (Carbomer), CARBOPOL® Ultrez 20 (Acrylates/C10-30 Alkyl Acrylates Copolymer), AVALURE® AC series (Acrylates Copolymer), AVALURER UR series (Polyurethane-2, Polyurethane-4, PPG-17/IPDI/DMPA Copolymer) polyethylene glycol; water-soluble acrylics; water-soluble polyesters; polyacrylamides; polyamines; polyquaternary amines; styrene maleic anhydride; (SMA) resin; polyethylene amine; and other conventional polymer that is polar solvent soluble or that can be made soluble through neutralization with the appropriate base.

The total amount of the one or more film-forming polymers in the mineral sunscreen composition, if present, will vary but may be from about 0.1 to about 15 wt. %, based on the total weight of the mineral sunscreen composition. In further embodiments, the mineral sunscreen composition includes from about 0.1 to about 10 wt. %, about 0.1 to about 8 wt. %, about 0.1 to about 5 wt. %, about 0.1 to about 3 wt. %, about 0.5 to about 15 wt. %, about 0.5 to about 10 wt. %, about 0.5 to about 8 wt. %, about 0.5 to about 5 wt. %, about 0.5 to about 3 wt. %, about 1 to about 15 wt. %, about 1 to about 10 wt. %, about 1 to about 8 wt. %, about 1 to about 5 wt. %, or about 1 to about 3 wt. %, based on the total weight of the mineral sunscreen composition.

### Stilbenoids

The mineral sunscreen compositions may optionally include one or more stilbenoids. Stilbenoids are secondary products of heartwood formations in trees that possess phytoalexin properties. In chemical terms, they are derivatives of stilbene, often containing one or more phenolic functional groups. In biochemical terms, they belong to the family of phenylalanine derivatives known as phenylpropanoids. Much of their biosynthetic pathway is shared with those of the aromatic chalconoids, such as chalcone.

A well-characterized botanical stilbenoid is resveratrol (3,5,4′-trihydroxy-trans-stilbene), a resorcinol derivative first isolated in 1939 from the white hellebore (Veratrum album), which is found in the skin of red grapes, and in other fruits and nuts including berries and peanuts. Nonlimiting examples of useful stilbenoids include piceid, resveratrol, piceatannol, pterostilbene, and a mixture thereof. In some instances, piceid and resveratrol are preferred stilbenoids; piceid being the most preferred.

The total amount of the stilbenoids in the mineral sunscreen compositions, if present, may be from about 0.01 to 8 wt. %, based on the total weight of the sunscreen composition. In further embodiments, the total amount of the one or more stilbenoids in the mineral sunscreen composition is from about 0.01 to about 5 wt. %, about 0.01 to about 3 wt. %, about 0.1 to about 8 wt. %, about 0.1 to about 5 wt. %, about 0.1 to about 3 wt. %, about 0.5 to about 8 wt. %, about 0.5 to about 5 wt. %, about 0.5 to about 3 wt. %, about 1 to about 8 wt. %, about 1 to about 5 wt. %, or about 1 to about 3 wt. %, based on the total weight of the mineral sunscreen composition.

### Photostabilizers

Photostabilizers are organic compounds that help to prevent UV filters from losing their effectiveness in sunlight. Certain photostabilizers help stabilizing UV filter molecules structurally and geometrically through electrostatic and van der Waals interactions, which makes them less likely to take part in chemical reactions. Other photostabilizers protect sunscreens by helping dissipating the energy from UV more quickly, thus reducing or even eliminating the possibility of a chemical reaction. This process is called energy transfer, and it can take place when the sunscreen agent and photostabilizer molecules exchange electrons. In this way, the sunscreen agents are fully active in protecting the skin by absorbing the harmful rays, while the photostabilizers dispose of the energy.

Nonlimiting examples of photostabilizers include alpha-cyanodiphenylacrylate, diesters or polyesters of naphthalene dicarboxylic acid, 4-hydroxybenzylidenemalonate derivatives or 4-hydroxycinnamate derivatives, 2-pyrrolidinone-4-carboxy ester, ethyhexyl methoxycrylene, diethylhexyl syringylidenemalonate, or combinations thereof.

In a preferred embodiment, the mineral sunscreen composition includes a combination photostabilizers selected from stilbenoids, and ethyhexyl methoxycrylene, diethylhexyl syringylidenemalonate. Stilbenoids, ethylhexylmethoxycrylene, and diethylhexyl syringylidenemalonate can synergistically interact in combination to unexpectedly boost the SPF of mineral UV filtering agents. Thus, the amount of stilbenoids, ethylhexylmethoxycrylene, and diethylhexyl syringylidenemalonate and the weight ratio of these compounds with respect to each other in the sunscreen compositions are sufficient to improve the SPF of the sunscreen composition.

In one embodiment, the one or more stilbenoids, ethylhexylmethoxycrylene, and diethylhexyl syringylidenemalonate are in amounts sufficient to boost the in vitro SPF of the sunscreen composition by at least 5% in comparison to an otherwise identical sunscreen composition in which the total amount of the stilbenoids, ethylhexylmethoxycrylene, and diethylhexyl syringylidenemalonate is replaced with only ethylhexylmethoxycrylene. In some cases, the in vitro SPF is boosted by at least 6%, at least 7%, at least 8%, at least 9%, or at least 10%.

In another embodiment, the one or more stilbenoids, ethylhexylmethoxycrylene, and diethylhexyl syringylidenemalonate are in amounts sufficient to boost the in vitro SPF of the sunscreen composition by at least 5% in comparison to an otherwise identical sunscreen composition in which the total amount of the stilbenoid(s), the ethylhexylmethoxycrylene, and the diethylhexyl syringylidenemalonate is replaced with only diethylhexyl syringylidenemalonate. In some cases, the in vitro SPF is boosted by at at least 8%, or at least 10%, at least 12%, at least 15%, at least 18%, or at least 20%.

The various amounts of the stilbenoids, ethylhexylmethoxycrylene, and diethylhexyl syringylidenemalonate can be useful for attaining the desired boost in SPF. Accordingly, in some embodiments, the amounts of the stilbenoids, ethylhexylmethoxycrylene, and diethylhexyl syringylidenemalonate are as follows:
about 0.1 to about 0.5, preferably about 0.1 to about 0.4, more preferably about 0.2 to about 0.4 of the one or more stilbenoids;
about 0.25 to about 0.75, preferably about 0.3 to about 0.7, more preferably about 0.3 to about 0.6 of of ethylhexylmethoxycrylene; and
about 0.1 to about 0.5, preferably, about 0.1 to about 0.45, more preferably about 0.15 to about 0.4 of diethylhexyl syringylidenemalonate.

The total amount of the combination of stilbenoids, ethylhexylmethoxycrylene, and diethylhexyl syringylidenemalonate in the sunscreen compositions can vary but is typically about 0.5 to about 10 wt. %, based on the total weight of the sunscreen composition. In some instances, the total amount of the combination of stilbenoids, ethylhexylmethoxycrylene, and diethylhexyl syringylidenemalonate in the sunscreen compositions may be about 0.5 to about 8 wt. %, about 0.5 to about 5 wt. %, about 0.5 to about 4 wt. %, about 0.5 to about 3 wt. %, about 1 to about 10 wt. %, about 1 to about 8 wt. %, about 1 to about 5 wt. %, about 1 to about 4 wt. %, or about 1 to about 3 wt. %, based on the total weight of the sunscreen composition. Regardless of the total amount of the stilbenoids, ethylhexylmethoxycrylene, and diethylhexyl syringylidenemalonate in the sunscreen compositions, the stilbenoids, ethylhexylmethoxycrylene, and diethylhexyl syringylidenemalonate may be present in the sunscreen composition according to the weight ratios set forth above.

### Fatty Alcohols

The mineral sunscreen composition may optionally include one or more fatty alcohols. Fatty alcohols typically include monohydric alcohols having 8-22 carbon atoms although longer chain alcohols in excess of 30 carbons may be used. The fatty alcohols may be saturated or unsaturated. The fatty alcohols may be straight or branched. In particular, the oil phase may comprise straight chain, saturated fatty alcohol with a terminal hydroxyl. Suitable fatty alcohols include decyl alcohol, lauryl alcohol, myristyl alcohol, cetyl alcohol, stearyl alcohol, isostearyl alcohol, cetearyl alcohol, icosyl alcohol, behenyl alcohol.

The total amount of one or more fatty alcohols, if present, will vary but may be in an amount of from about 0.1 to about 10 wt. %, based on the total weight of the mineral sunscreen composition. In further embodiments, the total amount of one or more fatty alcohols may be from about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, about 1 to about 5 wt. %, about 2 to about 8 wt. %, about 5 to about 10 wt. %, or about 5 to about 15 wt. %, based on the total weight of the mineral sunscreen compositions.

### Water Soluble Solvent

The mineral sunscreen composition can optionally include one or more water soluble solvents. The term “water soluble solvent” is interchangeable with the terms “water soluble organic solvent” and “water-miscible solvent” and means a compound that is liquid at 25° C. and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, organic solvents selected from glycerin, mono-alcohols (for example C₂₋₈, or C₂₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from alcohols (for example C₂₋₆ or C₂₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, and propane diol.

Further non-limiting examples of water soluble organic solvents include alkanediols (polyhydric alcohols) such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof. In a preferred embodiment, the composition include one or more glycols selected from propylene glycol, butylene glycol, pentylene glycol, hexylene glycol, caprylyl glycol, dipropylene glycol, and mixtures thereof.

The total amount of the one or more water soluble solvents in the mineral sunscreen composition, if present, will vary but may be from about 0.1 to about 20 wt. %, based on the total weight of the mineral sunscreen composition. In further embodiments, the mineral sunscreen composition may include from about 0.1 to about 15 wt. %, about 0.1 to about 10 wt. %, about 0.1 to about 5 wt. %, about 1 to about 20 wt. %, about 1 to about 15 wt. %, about 1 to about 10 wt. %, or about 1 to about 5 wt. % of one or more water soluble solvents, based on the total weight of the mineral sunscreen composition.

### Vitamins

The mineral sunscreen composition may optionally include one or more skin vitamins. “Vitamins” means vitamins, pro-vitamins, and their salts, isomers and derivatives. Non-limiting examples of suitable vitamins include vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

The total amount of one or more vitamins, if present, will vary but may be from about 0.01 to about 8 wt. %, based on the total weight of the mineral sunscreen composition. In further embodiments, the mineral sunscreen composition may include from about 0.01 to about 5 wt. %, about 0.01 to about 3 wt. %, about 0.1 to about 8 wt. %, about 0.1 to about 5 wt. %, or about 0.1 to about 3 wt. % of one or more vitamins, based on the total weight of the mineral sunscreen composition.

### Antioxidants

The mineral sunscreen compositions may optionally include one or more antioxidants. Suitable antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate, ascorbyl polypeptide and ascorbyl phosphate).

Oil-soluble antioxidants suitable for use in the compositions of this invention include, but are not limited to, butylated hydroxytoluene, retinoids (e.g., retinol and retinyl palmitate), tocopherols (e.g., tocopherol acetate), sodium tocopheryl phosphate, tocotrienols, alkylresorcinols, curcurmin and its derivatives and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, Phyllanthus emblica and propolis.

The total amount of one or more antioxidants, if present, will vary, but may be from about 0.1 to about 8 wt. %, based on the total weight of the mineral sunscreen composition. In further embodiments, the mineral sunscreen composition may include from about 0.01 to about 5 wt. %, about 0.01 to about 3 wt. %, about 0.1 to about 8 wt. %, about 0.1 to about 5 wt. %, or about 0.1 to about 3 wt. % of one or more antioxidants, based on the total weight of the mineral sunscreen composition.

### Miscellaneous Ingredients

The mineral sunscreen compositions may optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of miscellaneous ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the mineral sunscreen composition, if present, will vary but may be in an amount from about 0.1 to about 15 wt. %, based on the total weight of the mineral sunscreen composition. In further embodiments, the mineral sunscreen composition includes about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. % of one or more miscellaneous ingredients, based on the total weight of the mineral sunscreen composition.

### Ratio of Aqueous and Oil Phase

The mineral sunscreen compositions are in the form of an oil-in-water emulsions having an aqueous phase and an oil phase. The weight ratio of the oil phase to the aqueous phase will vary but can be from about 1:5 to about 5:1. In further embodiments, the weight ratio is from about 1:5 to about 1:2, about 1:5 to about 1:1, about 1:5 to about 1:2, about 1:4 to about 4:1, about 1:4 to about 2:1, about 1:4 to about 1:1, about 1:4 to about 1:2, about 1:3 to about 3:1, about 1:3 to about 1:1, about 1:2 to about 2:1 (oil phase: aqueous phase). In various embodiments, the may be a higher aqueous phase than oil phase.

### Aqueous Phase

The mineral sunscreen compositions are in the form of an oil-in-water emulsions having an aqueous phase and an oil phase. The aqueous phase may constitute about 10 to about 90 wt. % of the mineral sunscreen composition, based on the total weight of the mineral sunscreen composition. In further embodiments, the aqueous phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. %, based on the total weight of the mineral sunscreen composition.

### Oil Phase

The amount of the mineral sunscreen composition that is not the oil phase is the aqueous phase. The oil phase may constitute about 10 to about 90 wt. % of the mineral sunscreen composition, based on the total weight of the mineral sunscreen composition. In further embodiments, the oil phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. %, based on the total weight of the mineral sunscreen composition.

### Droplet Size

In various embodiments, the average droplet size of the oil phase in the emulsions forming the mineral sunscreen compositions is from about 10 nm to about 2 μm, about 10 nm to about 1.5 μm, about 10 nm to about 1 μm, about 10 nm to about 800 nm, about 10 nm to about 600 nm, about 10 nm to about 500 nm, about 10 nm to about 250 nm, about 50 nm to about 2 μm, about 50 nm to about 1.5 μm, about 50 nm to about 1 μm, about 50 nm to about 800 nm, about 50 nm to about 600 nm, about 50 nm to about 500 nm, about 50 nm to about 250 nm, about 100 nm to about 2 μm, about 100 nm to about 1.5 μm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

Droplet size can be determined using Brookhaven Dynamic Light Scattering (DLS). DLS is a technique used to determine droplet size in a colloidal system or emulsion. When the samples are illuminated with a monochromatic laser beam, the particles in the samples undergo Brownian motion, causing fluctuations in scattered light intensity. The scattered light is then collected at various angles, and the autocorrelation function of these intensity fluctuations is analyzed. The analysis provides information about the rate of diffusion of the particles, and from this, the size distribution is inferred using mathematical models. Brookhaven's DLS instruments use advanced algorithms to accurately interpret the data, offering insights into the dynamic behavior and size characteristics of particles ranging from a few nanometers to several micrometers in a liquid medium.

### In Vitro SPF

SPF (sun protection factor) is a measure of how effective a sunscreen composition will be able to provide protection from ultraviolet B (UV-B) radiation. The chief cause of reddening and sunburn, UV-B rays tend to damage the epidermis, skin's outer layers. The in vitro SPF a can be determined using methods known in the art. Nonetheless, a preferable method for determining in vitro SPF is the method described in Fageon, L. et al. Int. J. Cosmetic Sci., 2009, 405-17, which is incorporated herein by reference in its entirety.

The SPF of the mineral sunscreen composition can be adjusted and increased or decreased as desired, for example, by modifying the amounts and combinations of UV filtering agents in the composition. Different degrees of SPF are desired for different purposes and skin types.

The SPF of the mineral sunscreen composition can be adjusted as desired. Accordingly, the mineral sunscreen composition may have an SPF from about 5 to about 100. In various embodiments, an SPF from about 5 to about 50, about 5 to about 25, about 10 to about 50, about 10 to about 15, about 50 to about 100, about 50 to about 70, or about 5 to about 10, may be desired.

### Methods

The instant disclosure relates to methods for protecting a keratinous substrate, especially the skin and/or hair, from UV radiation, and to methods of absorbing UV light, and to methods for preventing sunburn. Such methods comprise application of a sunscreen composition to the skin and/or hair and protecting the skin and/or hair from ultraviolet radiation. Additionally, methods for boosting the SPF of mineral UV filtering agents, methods for reducing whiteness of mineral sunscreen compositions, and methods for improving the transparency of mineral sunscreen compositions are encompassed.

### EMBODIMENTS

In a preferred embodiment, the mineral sunscreen composition comprises, consists essentially of, or consists of:
(a) about 0.1 to about 20 wt. %, preferably about 0.1 to about 15 wt. %, more preferably about 1 to about 10 wt. % of a hydrophobic polymer, which is a reaction product of a natural or food-derived oil and a methacrylate or acrylate polymer, wherein preferably,
   the hydrophobic polymer is a reaction product of: (a) (i) a natural or food-derived oil selected from linseed oil, sunflower oil, tung oil, fish oil, cottonseed oil, soybean oil, or combinations thereof, preferably linseed oil, and (a) (ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
   the hydrophobic polymer is the reaction product of linseed oil and poly (isobutyl methacrylate);
(b) of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 0.1 to about 30 wt. %, preferably about 1 to about 25 wt. %, more preferably about 5 to about 20 wt. % of one or more mineral UV filtering agents, wherein preferably the one or more mineral UV filtering agents are selected from titanium dioxide, zinc oxide, iron oxides, cerium oxides, zirconium oxides, and a mixture thereof;
(d) about 0.1 to about 15 wt. %, preferably about 0.5 to about 10 wt. %, more preferably about 1 to about 8 wt. % of one or more surfactants, preferably a plurality of surfactants; and
(e) water;
(f) optionally, one or more organic UV filtering agents;
(g) optionally, one or more film forming polymers; and
(h) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion; and
   the weight ratio of the oil phase to the aqueous phase is from about 1:5 to about 5:1, preferably about 1:5 to about 1:2, more preferably about 1:5 to about 1:1, (oil phase:aqueous phase).

The mineral sunscreen compositions are in the form of an oil-in-water emulsions having an aqueous phase and an oil phase, as noted above. The aqueous phase may constitute about 10 to about 90 wt. % of the mineral sunscreen composition, based on the total weight of the mineral sunscreen composition. In further embodiments, the aqueous phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. %, based on the total weight of the mineral sunscreen composition.

The oil phase may constitute about 10 to about 90 wt. % of the mineral sunscreen composition, based on the total weight of the mineral sunscreen composition. In further embodiments, the oil phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. %, based on the total weight of the mineral sunscreen composition.

The pH of the sunscreen compositions may be from about 4.5 to about 8.5. In further embodiments, the pH of the emulsion may be from about 5 to about 8, about 5 to about 7, about 5 to less than 7, about 5.5 to about 6.5, or about 6 to about 7.5.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

In a preferred embodiment, the mineral sunscreen composition may have a smaller oil phase, wherein the sunscreen composition comprises, consists essentially of, or consists of:
(a) about 0.1 to about 8 wt. %, preferably about 0.1 to about 5 wt. %, more preferably about 0.5 to about 3 wt. % of a hydrophobic polymer, which is a reaction product of a natural or food-derived oil and a methacrylate or acrylate polymer, wherein preferably,
   the hydrophobic polymer is a reaction product of: (a)(i) a natural or food-derived oil selected from linseed oil, sunflower oil, tung oil, fish oil, cottonseed oil, soybean oil, or combinations thereof, preferably linseed oil, and (a)(ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
   the hydrophobic polymer is the reaction product of linseed oil and poly (isobutyl methacrylate);
(b) about 0.1 to about 10 wt. %, preferably about 0.1 to about 6 wt. %, more preferably about 0.5 to about 5 wt. % of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) one or more mineral UV filtering agents, wherein preferably the one or more mineral UV filtering agents are selected from titanium dioxide, zinc oxide, iron oxides, cerium oxides, zirconium oxides, and a mixture thereof;
(d) about 0.1 to about 15 wt. %, preferably about 0.5 to about 10 wt. %, more preferably about 1 to about 8 wt. % of one or more surfactants, preferably a plurality of surfactants;
(e) water;
(f) optionally, one or more organic UV filtering agents;
(g) optionally, one or more film forming polymers; and
(h) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

The pH of the sunscreen compositions may be from about 4.5 to about 8.5. In further embodiments, the pH of the emulsion may be from about 5 to about 8, about 5 to about 7, about 5 to less than 7, about 5.5 to about 6.5, or about 6 to about 7.5.

In another preferred embodiment, the mineral sunscreen composition comprises, consists essentially of, or consists of:
(a) about 0.1 to about 8 wt. %, preferably about 0.5 to about 5 wt. %, more preferably about 1 to about 5 wt. % of a hydrophobic polymer which is the reaction product of linseed oil and poly (isobutyl methacrylate);
(b) about 0.1 to about 10 wt. %, preferably about 0.1 to about 6 wt. %, more preferably about 0.5 to about 5 wt. % of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 1 to about 30 wt. %, preferably about 5 to about 25 wt. % of one or more mineral UV filtering agents, wherein preferably the one or more mineral UV filtering agents are selected from titanium dioxide, zinc oxide, iron oxides, cerium oxides, zirconium oxides, or mixtures thereof;
(d) about 0.1 to about 15 wt. %, preferably about 0.5 to about 10 wt. %, more preferably about 1 to about 8 wt. % of one or more surfactants, preferably a plurality of surfactants; and
(e) water;
(f) optionally, one or more organic UV filtering agents;
(g) optionally, one or more film forming polymers; and
(h) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

The pH of the sunscreen compositions may be from about 4.5 to about 8.5. In further embodiments, the pH of the emulsion may be from about 5 to about 8, about 5 to about 7, about 5 to less than 7, about 5.5 to about 6.5, or about 6 to about 7.5.

In another preferred embodiment, the mineral sunscreen composition comprises, consists essentially of, or consists of:
(a) about 0.1 to about 20 wt. %, preferably about 1 to about 55 wt. %, more preferably about 1 to about 10 wt. % of a hydrophobic polymer which is the reaction product of linseed oil and poly (isobutyl methacrylate);
(b) about 0.1 to about 50 wt. %, preferably about 1 to about 50 wt. %, more preferably about 20 to about 5 wt. % one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}}$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 1 to about 30 wt. %, preferably about 5 to about 25 wt. % of one or more mineral UV filtering agents, wherein preferably the one or more mineral UV filtering agents are selected from titanium dioxide, zinc oxide, iron oxides, cerium oxides, zirconium oxides, or mixtures thereof;
(d) about 0.1 to about 15 wt. %, preferably about 0.5 to about 10 wt. %, more preferably about 1 to about 8 wt. % of one or more surfactants, preferably a plurality of surfactants;
(e) water;
(f) optionally, one or more organic UV filtering agents;
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %; and
(h) optionally, one or more film-forming polymers;
   wherein the weight ratio of the oil phase to the aqueous phase is from about 1:5 to about 5:1, preferably about 1:5 to about 1:2, more preferably about 1:5 to about 1:1, (oil phase: aqueous phase).

The mineral sunscreen compositions are in the form of an oil-in-water emulsions having an aqueous phase and an oil phase, as noted above. The aqueous phase may constitute about 10 to about 90 wt. % of the mineral sunscreen composition, based on the total weight of the mineral sunscreen composition. In further embodiments, the aqueous phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. %, based on the total weight of the mineral sunscreen composition.

The oil phase may constitute about 10 to about 90 wt. % of the mineral sunscreen composition, based on the total weight of the mineral sunscreen composition. In further embodiments, the oil phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. %, based on the total weight of the mineral sunscreen composition.

The pH of the sunscreen compositions may be from about 4.5 to about 8.5. In further embodiments, the pH of the emulsion may be from about 5 to about 8, about 5 to about 7, about 5 to less than 7, about 5.5 to about 6.5, or about 6 to about 7.5.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

### EXAMPLES

Implementation of the present disclosure is provided by way of the following examples. The examples serve to illustrate the technology without being limiting in nature.

### Example 1

### Water-Resistance

The emulsions shown in the table below were prepared.

| | | A | B | C | D | E |
|---|---|---|---|---|---|---|
| | | wt. % | wt. % | wt. % | wt. % | wt. % |
| (a) | MYCELX ®¹ | 1 | 1 | 1 | | |
| (b) | CAPRYLIC/CAPRIC | 0.4 | 0.4 | 0.4 | 0.4 | 0.4 |
| | TRIGLYCERIDE | | | | | |
| (c) | M-RHAMNOLIPID | 2 | 2 | 2 | 2 | 2 |
| | SODIUM COCOYL | 0.6 | 0.6 | 0.6 | 0.6 | 0.6 |
| | TAURATE | | | | | |
| (d) | TiO₂-UV GRADE MT100 | 5 | 8 | 10 | 5 | 10 |
| (e) | WATER-QS | QS | QS | QS | QS | QS |

¹Reaction product of linseed oil and isobutyl methacrylate polymer

The compositions were prepared by mixing the m-rhamnolipid, sodium methyl cocoyl taurate, MycelX®, caprylic/capric triglyceride, and a small amount of water at 2,750 rpm for 2 minutes at 25° C. The composition is a homogenous mixture. The titanium dioxide was added to the homogenous mixture in different concentrations and additional water added to form the final emulsion having 5 wt. %, 8 wt. %, and 10 wt. % of titanium dioxide.

Inventive composition A and comparative composition D were used to determine the compositions resistance to water and washing with water. An amount of 0.5 ml of inventive composition A and comparative composition D were applied to glass slides and allowed to dry. The whiteness of the slides was assessed using a DigiEye® Color Measurement spectrometer, which determines L*a*b* values. The “L” value relates to whiteness. The glass slides were then washed with tap water at 40° C. and a low flow rate (500 ml/min), and allowed to dry. The whites of the slides was again assessed using the DigiEye® Color Measurement spectrometer.

Whiteness (L-Value) of Compositions on Glass

| | A | D |
|---|---|---|
| L-Value (whiteness) on glass slide dry | 93.20 | 92.70 |
| condition before washing | | |
| L-Value (whiteness) on glass slide dry | 89.18 | 27.71 |
| condition after washing | | |

The whiteness of inventive composition A and comparative composition D on the glass slides prior to washing was nearly identical. After the slides were washed, however, the glass slide treated with inventive composition A was significantly whiter than the glass slide treated with inventive composition D. This illustrates that inventive composition A is much more water-resistant than comparative composition D.

### Example 2

### Skin Transparency

An amount of 0.5 ml of inventive composition A and comparative composition D were (shown in Example 1) were uniformly applied to the back of hands. Inventive composition A was applied to the back of a left hand of an individual and comparative composition D was applied to the back of the right hand of the same individual. Both inventive composition A and comparative composition D were uniformly applied to the back of the hands and allowed to dry for 15 minutes.

The whiteness of the backs of the hands was assessed using a DigiEye® Color Measurement spectrometer, which determines L*a*b* values. The “L” value relates to whiteness. The results are shown in the table below.

Whiteness (L-Value) of Compositions on Skin

| | A | D |
|---|---|---|
| L-Value (whiteness) on | 64.16 | 75.15 |
| skin, dry condition before | | |
| washing | | |

The results show that skin treated with inventive composition A was less white (more transparent) than skin treated with comparative composition D, even though both compositions included the same amount of titanium dioxide.

### Example 3

### Skin Transparency and Water-Resistance

An amount of 0.5 ml of inventive composition C and comparative composition E were (shown in Example 1) were uniformly applied to the back of hands. Inventive composition C was applied to the back of a left hand of an individual and comparative composition E was applied to the back of the right hand of the same individual. Both inventive composition C and comparative composition E were uniformly applied to the back of the hands. The whiteness of the backs of the hands was assessed using a DigiEye® Color Measurement spectrometer, which determines L*a*b* values, while the composition were still wet. The “L” value relates to whiteness.

The compositions were allowed to dry on the back of the hands. After drying for 15 minutes, L-Values were again assessed.

The backs of the hands were then subsequently washed with tap water at 40° C. and low flow rate (500 ml/min). After washing, the backs of the hands were again assessed using the DigiEye® Color Measurement spectrometer while the hands were still wet. The results are shown in the table below.

Whiteness (L-Value) of Compositions on Skin

| | C | E |
|---|---|---|
| L-Value (whiteness) on | 77.40 | 78.28 |
| skin, wet condition before | | |
| washing | | |
| L-Value (whiteness) on | 73.17 | 76.15 |
| skin, dry condition before | | |
| washing | | |
| L-Value (whiteness) on | 76.90 | 71.16 |
| skin, wet condition after | | |
| washing | | |

The whiteness of inventive composition C and comparative composition E on the skin prior after initial application before drying was essentially identical. Upon drying, however, inventive composition C was less white (more transparent) than comparative composition E. After rinsing, inventive composition C was more white (less transparent) than comparative composition E. This shows that inventive composition C is more water-resistant than comparative composition E. However, inventive composition C is more transparent after drying on the skin than comparative composition E, when equal amounts of titanium dioxide are applied to the skin.

### Example 4

### Water-Resistance and SPF

The mineral sunscreen compositions shown in the table below were prepared and tested for their water-proof properties.

| | Control | Inventive | | Comparative |
|---|---|---|---|---|
| | F | G | H | I |
| | wt. % | wt. % | wt. % | wt. % |
| MycelX ®¹ | | 2.5 | 2.5 | |
| Rhamnolipid | | | 2.7 | |
| Sodium Methyl Cocoyl Taurate | | | 0.9 | |
| Soja Glyceride | | | | 2.5 |
| Steareth-20, Steareth-2, Cetearyl | 3.6 | 3.6 | | 3.6 |
| Alcohol, Glyceryl Stearate, and | | | | |
| Polysorbate-20 | | | | |
| C15-19 Alkane, | 7 | 6 | 6 | 6 |
| Cetearyl Isononanoate | 3.5 | 3 | 3 | 3 |
| Dicaprylyl Carbonate | 6.5 | 5.5 | 5.5 | 5.5 |
| Caprylyl Glycol and Glycerin | 5.3 | 5.3 | 5.3 | 5.3 |
| Zinc Oxide (and) | 20.3 | 20.3 | 20.3 | 20.3 |
| Triethoxycaprylylsilane, Titanium | | | | |
| Dioxide (and) Isohexadecane (and) | | | | |
| Triethylhexanoin (and) Aluminum | | | | |
| Stearate (and) Alumina, | | | | |
| Polyhydroxystearic Acid | | | | |
| Acrylates/C10-30 Alkyl Acrylate | 0.3 | 0.3 | 0.3 | 0.3 |
| Crosspolymer | | | | |
| Xanthan Gum | 0.2 | 0.2 | 0.2 | 0.2 |
| Tocopherol and | 2.8 | 2.8 | 2.8 | 2.8 |
| Chlorphenesin, Polyhydroxstearic | | | | |
| Acid, Hydroxyacetophenone | | | | |
| Water QS to 100 | QS | QS | QS | QS |

¹Reaction product of linseed oil and isobutyl methacrylate polymer

An amount of 1.3 mg/cm² of each composition from the table above was applied to polymethylmethacrate (PMMA) plates (HD6) using a robot and the plates allowed to dry for about 15-30 minutes (until totally dry). An initial Sun Protection Factor (SPF) was then measured at 64 locations across the PMMA plates. The plates were then positioned vertically in brackets of a Dissolution Apparatus, which immerses the plates halfway into deionized (DI) water and stirs the plate at 24 rpm at 25° C. for 40 minutes. The plates were removed and allowed to dry vertically for about 30-40 minutes (until totally dry). The SPF was measured again across the plates using an 8×8 grid at 64 locations maintaining the same orientation used for the initial measurements. The percentage of water resistance was then calculated as follows.

$$WR \% = \frac{{SPF}_{final} - 1}{{SPF}_{initial} - 1} \times 100 \%$$

The lowest percentage of water resistance appears along the bottom row of the plates, with an average percentage of water resistance as shown in the table below.

| | | |
|---|---|---|
| Control | Inventive | Comparative |

| F | G | H | I |
|---|---|---|---|
| 14.47% | 31.10% | 34.99% | 13.34% |

The inventive sunscreen compositions provided more than double the percentage water resistance compared to both the control and the comparative sun screen composition.

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the applications or uses thereof. Accordingly, the description is not intended to limit the invention to the form disclosed herein. Also, it is intended to the appended claims be construed to include alternative embodiments.

Where the following terms are used in this specification, they are used as defined below.

The terms “comprising,” “having,” and “including” are used in their open, non-limiting sense.

The terms “a” and “the” are understood to encompass the plural as well as the singular.

The term “mineral UV filtering agent” is interchangeable with the terms “mineral UV screening agent,” “inorganic UV filtering agent,” “inorganic UV screening agent,” “mineral UV filter, and “inorganic UV filter.” Mineral UV filtering agents are compounds that do not include any carbon atoms in their chemical structures that are capable of screening out or absorbing UV radiation between 280 and 400 nm.

The compositions and methods of the present disclosure can comprise, consist of, or consist essentially of the essential elements and limitations of the disclosure described herein, as well as any additional or optional ingredients, components, or limitations described herein or otherwise useful.

All percentages, parts and ratios herein are based upon the total weight of the compositions of the present disclosure, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Other than in the operating examples, or where otherwise indicated, all numbers expressing quantities of ingredients and/or reaction conditions are to be understood as being modified in all instances by the term “about,” meaning within +/−5% of the indicated number.

As used herein, the expression “at least one” is interchangeable with the expression “one or more” and thus includes individual components as well as mixtures/combinations.

The term “treat” (and its grammatical variations) as used herein refers to the application of compositions of the present disclosure onto the surface of skin and/or hair. The term ‘treat” (and its grammatical variations) as used herein also refers to contacting the skin or hair with the compositions of the present disclosure.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, less than 0.01 wt. %, or none of the specified material.

The term “active material” as used herein with respect to the percent amount of an ingredient or raw material, refers to 100% activity of the ingredient or raw material.

“Cosmetically acceptable” means that the item in question is compatible with a keratinous substrate such as skin and hair. For example, a “cosmetically acceptable carrier” means a carrier that is compatible with a keratinous substrate such as skin and hair.

The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is non-limiting.

The phrase “stable emulsion” refers to a composition that does not undergo phase separation up to a temperature of 45° C. for at least two weeks.

The expression “inclusive” for a range of concentrations means that the limits of the range are included in the defined interval.

“Volatile”, as used herein, means having a flash point of less than about 100° C. “Non-volatile”, as used herein, means having a flash point of greater than about 100° C.

The term “polymers,” as defined herein, include homopolymers and copolymers formed from at least two different types of monomers.

The term “INCI” is an abbreviation of International Nomenclature of Cosmetic Ingredients, which is a system of names provided by the International Nomenclature Committee of the Personal Care Products Council to describe personal care ingredients.

The term “weight ratio” or “mass ratio” as used herein, references the amount of a substance in proportion to a mixture containing said substance, and is calculated by dividing the amount of said substance by weight contained in the mixture by the weight of the mixture containing said substance. As an example, a weight ratio of 0.4 for substance A in a mixture of A, B, and C indicates that the weight of substance A divided by the total weight of substances A, B, and C is 0.4.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular composition includes both a nonionic surfactant and a fatty compound, a single fatty acid will serve as only the nonionic surfactant or as only the fatty compound (the single fatty acid does not serve as both the nonionic surfactant and the fatty compound).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A mineral sunscreen composition comprising:
(a) a hydrophobic polymer formed as a reaction product of a natural or food-derived oil and a methacrylate or acrylate polymer;
(b) one or more solvents capable of solubilizing the reaction product of (a);
(c) one or more mineral UV filtering agents;
(d) one or more surfactants; and
(e) water;
wherein the composition is an oil-in-water emulsion.

2. The composition of claim 1, wherein the hydrophobic polymer is the reaction product formed from a natural or food-derived oil and a methacrylate polymer.

3. The composition of claim 1, wherein the natural or food-derived oil is a drying oil or semi-drying oil selected from linseed oil, sunflower oil, tung oil, fish oil, cottonseed oil, soybean oil, or combinations thereof.

4. The composition of claim 1, wherein the polymer is derived from monomers selected from isobutyl methacrylate, methyl methacrylate, ethyl methacrylate, n-butyl methacrylate, and combinations thereof.

5. The composition of claim 1, wherein the hydrophobic polymer is the reaction product of about 50 to about 85 parts by weight of the natural or food-derived oil and about 15 to about 50 parts by weight of the methacrylate or acrylate polymer.

6. The composition of claim 1, wherein the hydrophobic polymer is the reaction product of about 72 to about 77 parts by weight of the natural or food-derived oil and about 23 to about 28 parts by weight of a methacrylate polymer.

7. The composition of claim 1, wherein the polymer is isobutyl methacrylate polymer.

8. The composition of claim 1, wherein the hydrophobic polymer is the reaction product of linseed oil and poly (isobutyl methacrylate).

9. The composition of claim 1, wherein the one or more solvents capable of solubilizing the reaction product of (a) have a dispersion component (D), a polar component (P), a hydrogen bonding component (H), and a distance (Ra) of less than or equal to 13.4 MPa^{0.5} per Hansen Solubility Parameters, wherein the distance (Ra) is defined by formula (I):
$$Ra = \sqrt{4 {( D - {D}_{1} )}^{2} + {( P - {P}_{1} )}^{2} + {( H - {H}_{1} )}^{2}} \qquad ( I )$$
wherein
D₁ is 16.8 MPa^{0.5},
P₁ is 4.8 MPa^{0.5}, and
H₁ is 13.0 MPa^{0.5}.

10. The composition of claim 1, wherein at least one of the one or more solvents capable of solubilizing the reaction product of (a) is selected from polycitronellol acetate, caprylic/capric triglyceride, isododecane, isohexadecane, tetradecane, isopropyl myristate, isopropyl alcohol, octyldodecanol, ethanol, phenoxyethanol, castor oil, or mixtures thereof.

11. The composition of claim 1, wherein the one or more mineral UV filtering agents are selected from titanium dioxide, zinc oxide, iron oxides, cerium oxides, zirconium oxides, and a mixture thereof.

12. The sunscreen composition of claim 1, wherein the sunscreen composition is free from organic UV filtering agents.

13. The sunscreen composition of claim 1, wherein the total amount of the one or more inorganic UV filtering agents is from about 1 to about 25 wt. %, based on the total weight of the sunscreen composition.

14. The composition of claim 1, wherein the one or more biosurfactants are selected from glycolipids, wherein the glycolipids are selected from sophorolipids, rhamnolipids, trehalose lipids, mannosylerythritol lipids, or combinations thereof.

15. The composition of claim 1, wherein the one or more surfactants are selected from anionic surfactants, nonionic surfactants, amphoteric surfactants, or combinations thereof.

16. The composition of claim 15 comprising one or more anionic surfactants, wherein the one or more anionic surfactants are selected from acyl glutamates, acyl taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates, or combinations thereof.

17. The composition of claim 16 comprising one or more anionic surfactants, wherein at least one of the one or more anionic surfactants is an acyl taurate.

18. The composition of claim 1, wherein the oil-in-water emulsion is a dispersion having a droplet size from about 10 nm to about 1 μm.

19. A mineral sunscreen composition comprising:
(a) about 0.5 to about 10 wt. % of a hydrophobic polymer resulting from a reaction product of a natural or food-derived oil selected from linseed oil, sunflower oil, tung oil, fish oil, cottonseed oil, soybean oil, and combinations thereof and a polymer formed from one or more monomers selected from isobutyl methacrylate, methyl methacrylate, ethyl methacrylate, and n-butyl methacrylate and combinations thereof;
(b) about 0.1 to about 10 wt. % of one or more solvents capable of solubilizing the reaction product of (a);
(c) one or more mineral UV filtering agents;
(d) about 1 to about 10 wt. % of a plurality of surfactants, wherein the plurality of surfactants comprises:
(c)(i) about 0.1 to about 8 wt. % of one or more rhamnolipids; and
(c)(ii) about 0.1 to about 8 wt. % of one or more additional surfactants selected from anionic surfactants, nonionic surfactants, amphoteric surfactants, and cationic surfactants, or mixtures thereof; and
(d) water;
wherein the composition is an oil-in-water emulsion having a droplet size of about 10 nm to about 2 μm, and
all percentages by weight are based on a total weight of the composition.

20. A method for protecting skin and hair from UV radiation comprising applying a sunscreen composition of claim 1 to the skin or hair.
